(19) United States
(12) Patent Application Publication
(10) Pub. No.: US 2004/0133219 A1
(43) Pub. Date: Jul. 8, 2004

(54) Multi-material constriction device for forming stoma opening

(76) Inventor: Peter Forsell, Zug (CH)

Correspondence Address: NIXON & VANDERHYE, PC, 1100 N GLEBE ROAD, 8TH FLOOR, ARLINGTON, VA, 22201-4714, US

(21) Appl. No.: 10/623,801

(22) Filed: Jul. 22, 2003

Related U.S. Application Data
(60) Provisional application No. 60/398,810, filed on Jul. 29, 2002.

(51) Int. Cl. A61F 2/04

(52) U.S. Cl. 606/151; 623/23.65

(57) ABSTRACT

An implantable constriction device for forming a restricted stoma opening in the stomach or esophagus of a patient comprises an elongate composite structure adapted to constrict the stomach or esophagus of the patient. The elongate composite structure is composed of a base material, such as hard silicone, making the composite structure self-supporting. Property improving means is provided for improving at least one physical property of the composite structure other than self-supporting properties, such as fatigue resistance, liquid impermeability, aggressive body cells resistance, anti-friction properties and lifetime.

This application claims the benefit of Provisional Application No. 60/398,810, filed Jul. 29, 2002, the entire contents of which is hereby incorporated by reference in this application.

### BACKGROUND OF THE INVENTION

The present invention relates to an implantable constriction device for forming a stoma opening in the stomach or esophagus of a patient, for treating obesity or reflux and heartburn disease.

This kind of constriction device, in the form of a gastric banding device, in which a band encircles and constricts a portion of a patient's stomach to restrict the food intake of the patient, has been used in surgery for morbid obesity to form a small gastric pouch above the band and a reduced stoma opening in the stomach. Although such a band is applied around the stomach to obtain an optimal stoma opening during surgery, some prior gastric banding devices are provided with an adjustment device enabling a minor post-operation adjustment of the size of the stoma opening. For example, U.S. Pat. No. 6,067,991 discloses a mechanically adjusted gastric band and U.S. Pat. No. 6,102,922 discloses a hydraulically adjusted gastric band having an inflatable cavity. In practice, the band is made of silicone, which is a material approved and widely used for implantation. Moreover, the silicone band has an acceptable tensile strength and is fairly resistant to aggressive body fluids. Where the band is hydraulically adjusted the hydraulic fluid used typically is an isotonic salt solution mixed with other conventional materials.

A problem with traditional silicone gastric bands, however, is that the silicone material gives the band certain inadequate properties, such as poor fatigue resistance and poor endurance of static bending forces, which over time might result in breakage of the band. Furthermore, silicone is a material that is semi-permeable by liquid, which is a drawback to hydraulic silicone bands, because hydraulic fluid can escape by diffusing through the silicone material. As a result, accurate adjustments of a hydraulic band are difficult to perform because of the loss of hydraulic fluid and the need for a patient to regularly visit a doctor to add hydraulic fluid to and calibrate the constriction device. These inadequate properties are serious, considering that the band is implanted for the rest of the patient's life. Another problem is that the band somewhat restrains the dynamics movements of the stomach necessary for digesting the food. As a consequence, the band might erode the stomach wall so that the stomach wall becomes thinner over time, which dramatically increases the risk of the band penetrating the stomach wall.

The kind of constriction device presented initially has also been used for treating heartburn and reflux disease due to hiatal hernia, i.e. a portion of the stomach immediately below the gastric fundus slides upwardly through the esophageal hiatus. A consequence of hiatal hernia is that stomach acids and foods are regurgitated into the esophagus. For example, WO 01/49245 A2 discloses a mechanically adjusted silicone band of a constriction device that constricts the esophagus (or the stomach close to the cardia) of a patient to close the esophagus between meals. Thus, in this case the constriction device functions as an artificial sphincter. WO 01/47435 A2 discloses a similar constriction device including a hydraulically adjusted silicone band also functioning as an artificial sphincter.

The same inadequate properties stated above in connection with gastric bands also apply for silicone bands designed for the treatment of heartburn and reflux disease. A specific problem of implanted bands for treating heartburn an reflux disease is that the movements of the esophagus occurring when the patient swallows food are somewhat restrained by the band. As a consequence, the band might erode and injure the esophagus.

### SUMMARY OF THE INVENTION

The object of the present invention is to provide a new implantable constriction device for forming a stoma opening having improved properties as compared to traditional constriction devices.

Another object of the invention is to provide a new implantable constriction device suited for treating obese patients as well as patients suffering from heartburn and reflux disease.

Accordingly, the present invention provides an implantable constriction device for forming a restricted stoma opening in the stomach or esophagus of a patient, the constriction device comprising an elongate composite structure adapted to constrict the stomach or esophagus to form the stoma opening therein, wherein the elongate composite structure is composed of a base material making the composite structure self-supporting and property improving means for improving at least one physical property of the composite structure other than self-supporting properties.

In accordance with a first embodiment of the invention, the property improving means comprises a coating on the base material at least along a side of the elongate composite structure that is intended to contact the stomach or esophagus, wherein the coating has better aggressive body fluid resistance than the base material. Such a coating may comprise a Teflon™ or Parylene™ coating, or a biocompatible metal coating such as gold, silver or titanium. As a result, the constriction device can be protected from damaging influences of aggressive body fluids, possibly for the rest of the patient's life.

Where traditional silicone material constitutes the base material, a Teflon™ or Parylene™ coating also provides the composite structure with better anti-friction properties than the base material. Good anti-friction properties of the composite structure reduce the risk of the elongate composite structure eroding the stomach or esophagus. This is proven by tests, in which the surface of traditional silicone bands has been polished before use. Accordingly, the test results indicate significant improvements in avoiding erosion of the stomach or esophagus.

Furthermore, the Teflon™, Parylene™ or metal coating also makes the composite structure, in which the base material is made of silicone, stronger than the traditional silicone band. A stronger band reduces the risk of fracture.

In one alternative to the first embodiment, the elongate composite structure is designed for mechanical adjustment, such as the mechanical solutions disclosed in International Application No. WO 01/45486. In this alternative, the property improving means comprises a core of a soft viscoelastic material, such as silicone gel, typically having a hardness less than 20 Shure, cellulose gel or collagen gel. Where silicone gel is chosen, it may be “Med 3-6300” manufactured by Nusil. Hard silicone constitutes the base material, typically having a hardness of at least 60 Shure, and covers the soft core of viscoelastic material. The soft core makes the implanted elongate composite structure less injurious to the stomach or esophagus, and reduces the injury of such organs. Furthermore, the soft core of viscoelastic material may be formed to enclose and protect mechanical adjustment components and other components of the composite structure, whereby fibrosis is prevented from growing into such components.

In another alternative to the first embodiment, the elongate composite structure is designed for hydraulic adjustment, such as the hydraulic solutions disclosed in International Application No. WO 01/50833. In this alternative, the base material forms a closed tubing, which can be inflated by adding hydraulic fluid to the interior of the tubing and deflated by withdrawing hydraulic fluid from the interior of the tubing. The coating of Teflon™, Parylene™ or metal may cover the inner surface of the tubing. The base material may form two coaxial tubular layers of hard silicon, and the property improving means may comprise a tubular intermediate layer of a soft viscoelastic material located between the coaxial tubular layers. Alternatively, the base material may form an outer tubular layer and an inner arcuate layer attached to the outer tubular layer, the outer and inner layers defining a curved space extending longitudinally along the tubing. The property improving means may comprise a viscoelastic material filling the space. The tubing is applied around the stomach or esophagus so that the space with viscoelastic material is located closest to the stomach or esophagus. The viscoelastic material gives the advantages that erosion of the stomach or esophagus is reduced and the risk of hydraulic fluid leaking from the tubing is decreased.

In accordance with a second embodiment of the invention, the base material forms a first layer and the property improving means comprises a second layer applied on the first layer, wherein the second layer is more fatigue resistant than the first layer. The first layer preferably is comprised of hard silicone, whereas the second layer preferably is comprised of a polyurethane layer. In a traditional silicone band, especially the tubular type, that is formed in a loop to constrict the stomach or esophagus, the inner surface of the band loop that contacts the stomach or esophagus forms bulges and creases that repeatedly change as the band is subjected to dynamic movements from the stomach or esophagus and when the size of the band is adjusted. As a consequence, the implanted traditional silicone band has the drawback that it may crack after some time due to fatigue of the silicone material. With the elongate composite structure of the invention, in which hard silicone may constitute the base material and a fatigue resistant polyurethane layer covers the silicone material on the side of the elongate composite structure that contacts the stomach or esophagus, this drawback is eliminated.

The property improving means suitably comprises a coating that may be coated on the layer of hard silicone and/or the layer of polyurethane, wherein the coating has better aggressive body fluid resistance properties and/or better anti-friction properties than hard silicone. As described above in connection with the first embodiment, the coating may comprise a Teflon™ or Parylene™ coating, or a biocompatible metal coating.

The layer of hard silicone may form an inflatable tubing and the layer of polyurethane may cover the hard silicone layer within the tubing.

In accordance with a third embodiment of the invention, the base material forms an inflatable tubing and the property improving means comprises a liquid impermeable coating coated on the base material. The coating may be coated on the external and/or internal surface of the tubing. Preferably, the liquid impermeable coating comprises a Parylener™ coating, or a biocompatible metal coating. Where hard silicone, which is a liquid semi-permeable material, constitutes the base material, the coating of Parylene™ or metal gives the advantage that the tubing may be inflated by hydraulic fluid under pressure without risking fluid diffusing through the silicone wall of the tubing.

Also, in the third embodiment, the base material may form two coaxial tubular layers of hard silicon, and the property improving means may comprise a tubular intermediate layer of a soft viscoelastic material located between the coaxial tubular layers. Alternatively, the base material may form an outer tubular layer of hard silicone and an inner arcuate layer of silicone attached to the outer tubular layer. The outer and inner layers define a curved space extending longitudinally along the tubing and filled with the viscoelastic material. The tubing is intended to be applied around the stomach or esophagus so that the space with viscoelastic material is located closest to the stomach or esophagus.

In accordance with a fourth embodiment of the invention, the property improving means comprises gas, such as air, contained in a multiplicity of cavities formed in the base material to improve the flexibility of the composite structure. In this case, Teflon™ advantageously constitutes the base material. The cavities may be defined by net structures of the Teflon™ material. Thus, the resulting composite structure of Teflon™ and cavities filled with gas is strong, flexible and aggressive body fluid resistant, and has good tensile strength and good anti-friction properties. Also, in the fourth embodiment, the elongate composite structure may comprise an inflatable tubing.

The present invention also provides an implantable constriction device for treating an incontinent patient, comprising an elongate composite structure adapted to constrict the stomach or esophagus of the patient, wherein the composite structure includes an elongate biocompatible self-supporting base material having surfaces exposed to aggressive body cells, when the constriction device is implanted in the patient, and a cell barrier coating on the surfaces to prevent body cells from breaking down the base material, which is typically silicone. If the base material were broken down by such body cells, typically macrophages or killer cells, histological particles would be spread in the human body.

The barrier coating may comprise a Parylene™ coating or a biocompatible metal coating.

Alternatively, the barrier coating may comprise a composite of different materials to achieve the cell-barrier protection as described above. There are several examples of such composite materials on the market, for example a composite of polyurethane and silicone called Elaston™.

### BRIEF DESCRIPTION OF THE DRAWINGS

FIG. 1 is a front view of a mechanical constriction device according to the present invention.

FIG. 2 is an enlarged cross-section along the line II-II in FIG. 1.

FIGS. 3 and 4 are modifications of the embodiment shown in FIG. 2.

FIG. 5 is a front view of a hydraulic constriction device of the invention.

FIG. 6 is an enlarged cross-section along the line VI-VI in FIG. 5.

FIGS. 7-10 are modifications of the embodiment shown in FIG. 6.

FIG. 11 is a modification of the embodiment shown in FIG. 2.

Referring to the drawing figures, like reference numerals designate identical or corresponding elements throughout the several figures.

### DETAILED DESCRIPTION OF THE DRAWINGS

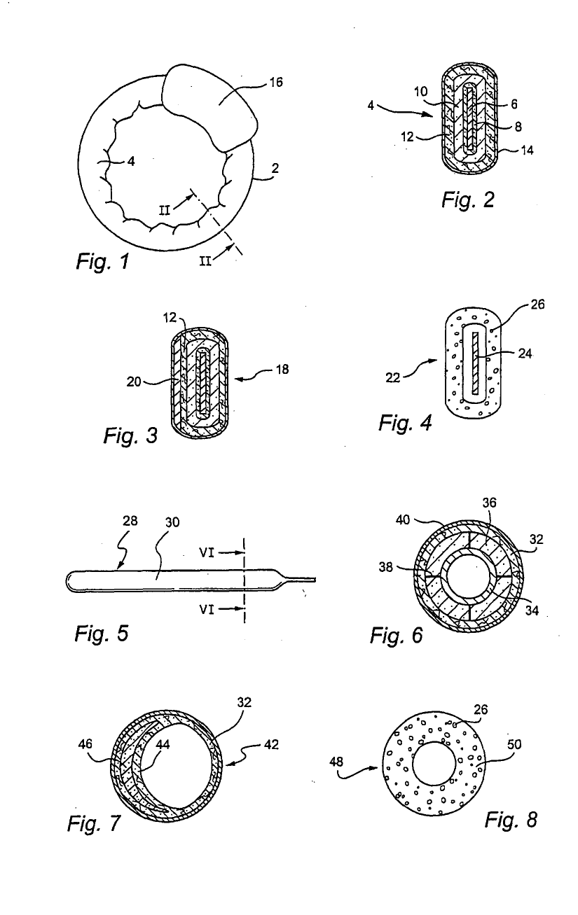

FIG. 1 illustrates a mechanical constriction device 2 according to the invention comprising an elongate composite structure 4 adapted to extend around and constrict the stomach or esophagus of a patient to form a restricted stoma opening therein. Referring to FIG. 2, the elongate composite structure 4 comprises a strong band 6 of nylon or the like, a tubular layer 8 of hard silicone, in which the band 6 slides, a soft layer 10 of a viscoelastic material, here a silicone gel having a hardness not more than 20 Shure, encircling the hard silicone layer 8, and a tubular layer 12 of a self-supporting base material of hard silicone having a hardness of at least 60 Shure, surrounding the soft silicon layer 10. A coating 14 of Teflon™, Parylene™ or a biocompatible metal, such as gold, silver or titanium, is coated on the outer hard silicone layer 12 to make the composite structure resistant to aggressive body fluids and to give the composite structure good anti-friction properties. A coating of Teflon™, Parylene™ or metal may also be coated on the internal surface of the inner tubular hard silicone layer 8 to reduce the friction between the nylon band 6 and the layer 8. The constriction device 2 has an adjustment means 16 that can displace the end portions of the nylon band 6 relative to each other to either enlarge or constrict the stoma opening.

FIG. 3 shows an elongate composite structure 18 similar to that of FIG. 2, except that a layer 20 of a fatigue resistant material, here polyurethane, is applied on the hard silicone layer 12 along the inner side of the structure 18 that is intended to contact the stomach or esophagus. Alternatively, the layer 20 may be tubular and surround the layer 12.

FIG. 4 shows a cross-section of an elongate composite structure 22 of an embodiment of the invention, in which Teflon™ constitutes the self-supporting base material, which is formed with a longitudinal cavity in which a strong nylon band 24 slides. Property improving means in the form of gas, here air, contained in a multiplicity of cavities 26 are formed in the base material to improve the flexibility thereof.

FIG. 5 shows a hydraulic constriction device 28 according to the invention comprising an elongate composite structure in the form of an inflatable tubing 30, in which the base material of hard silicone forms an outer tubular layer 32 and an inner coaxial layer 34. A viscoelastic material, here soft silicone gel, forms an intermediate layer 36 located between the tubular layers 32,34. Four longitudinal partition walls 38 between the tubular layers 32,34 divide the intermediate layer 36 into four sections to prevent the silicone gel from displacing in the circumferential direction of the tubing 30. (Also the embodiments according to FIGS. 2 and 3 may be provided with such longitudinal partition walls.) The outer layer 32 is coated with a coating 40 of Teflon™, Parylene™ or metal. Also the inner layer 34 may be coated with a coating of Teflon™, Parylene™ or metal. If a Parylene™ or metal coating is chosen the composite structure will be completely liquid impermeable.

FIG. 7 shows a tubing 42 similar to that of FIG. 6, except that an inner arcuate layer 44 is substituted for the inner tubular layer 34. The arcuate layer 44 is attached to the outer tubular layer 32, so that the outer tubular layer 32 and the arcuate layer 44 define a curved space extending longitudinally along the tubing 42. A viscoelastic material, here silicone gel 46, fills the space. In this embodiment there is no need for partition walls of the kind shown in the embodiment according to FIG. 6. The tubing 42 is intended to be applied around the stomach or esophagus so that the space with the protecting soft silicone gel 46 is located close to the stomach or esophagus.

As taught by the embodiment of FIG. 7, in the composite structures shown in FIGS. 2 and 3 the soft silicone gel may alternatively be applied in a longitudinal space close to the inner side of the elongate composite structure 4 and 18, respectively, that is intended to contact the stomach or esophagus.

In the same manner as described above in connection with the embodiment of FIG. 3, a layer of a fatigue resistant material, here polyurethane, may be applied on the outer tubular layer 32 of hard silicone of the tubing 30 and 42, respectively, along the side of the tubing 30 and 42, respectively, that is intended to contact the stomach or esophagus, when the tubing 30 and 42, respectively, encircles the stomach or esophagus.

FIG. 8 shows a cross-section of an elongate composite structure 48 of an embodiment of the invention, in which Teflon™ constitutes the self-supporting base material, which is formed to an inflatable tubing 50. Property improving means in the form of gas contained in a multiplicity of cavities 26 are formed in the base material to improve the flexibility of the tubing 50.

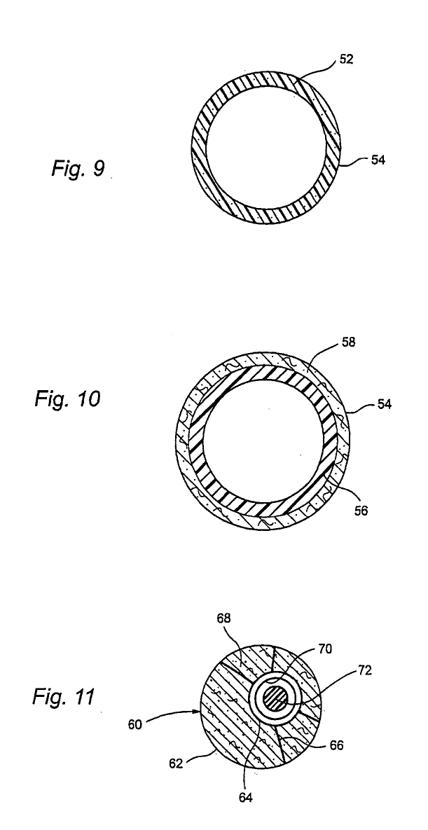

FIG. 9 shows a cross-section of a tubular composite structure of an embodiment of the invention, in which the self-supporting base material 52 is made of a polymer material suited for implantation, for example silicone or polyurethane. A property improving coating 54, for example made of Parylene™, Teflon™ or metal, is applied on the external surface or on both the external and internal surfaces of the tubular structure

FIG. 10 shows the same embodiment as FIG. 9 except that the base material comprises a layer 56 of polyurethane surrounded by a layer 58 of silicone.

FIG. 11 shows a cross-section of a mechanical constriction device of another embodiment of the invention, comprising a double walled tubing 60 of a self-supporting base material of hard silicone. The tubing 60 has an external wall 62 and an internal wall 64 spaced from the external wall 62, partition walls 66 dividing the space between the external and internal walls 62 and 64, respectively, of the tubing 60 into longitudinal cells 68, which are filled with a soft viscoelastic material, for example silicone gel. The internal wall 64 is coated with a friction reducing coating 70, for example made of Teflon™ or the like. A strong band 72 of nylon or the like slides in the tubing 60 on the friction reducing coating 70 to enable adjustment of the constriction device in the same manner as described above in connection with the embodiment according to FIGS. 1 and 2.

Although the present invention has been described in terms of particular embodiments, it is not intended that the invention be limited to those embodiments. Modifications of the embodiments within the spirit of the invention will be apparent to those skilled in the art. The scope of the invention is defined by the claims that follow.

What is claimed is:

1. An implantable constriction device for forming a restricted stoma opening in the stomach or esophagus of a patient, comprising an elongate composite structure adapted to constrict the stomach or esophagus of the patient, wherein said elongate composite structure is composed of a base material making said composite structure self-supporting and property improving means for improving at least one physical property of said composite structure other than self-supporting properties.

2. An implantable constriction device according to claim 1, wherein said property improving means comprises a coating on said base material at least along a side of said elongate composite structure that is intended to contact the stomach or esophagus, said coating having better aggressive body fluid resistant properties than said base material.

3. An implantable constriction device according to claim 2, wherein said coating is selected from the group consisting of a Teflon™, Parylene™, and a biocompatible metal coating.

4. An implantable constriction device according to claim 3, wherein the biocompatible metal coating is selected from the group consisting of gold, silver and titanium.

5. An implantable constriction device according to claim 2, wherein said property improving means comprises a core of a viscoelastic material covered with said self-supporting base material.

6. An implantable constriction device according to claim 5, wherein hard silicone constitutes said base material.

7. An implantable constriction device according to claim 5, wherein said viscoelastic material is selected from the group consisting of silicone gel, cellulose gel, and collagen gel.

8. An implantable constriction device according to claim 2, wherein said base material forms an inflatable tubing.

9. An implantable constriction device according to claim 8, wherein said tubing has an inner surface defining the interior of said tubing, and said coating covers said inner surface.

10. An implantable constriction device according to claim 8, wherein said coating is selected from the group consisting of Teflon™, Parylene™, and a biocompatible metal coating.

11. An implantable constriction device according to claim 10, wherein the biocompatible metal coating is selected from the group consisting of gold, silver and titanium.

12. An implantable constriction device according to claim 8, wherein hard silicone constitutes said base material.

13. An implantable constriction device according to claim 8, wherein said base material forms two coaxial tubular layers and said property improving means comprises a tubular intermediate layer of a viscoelastic material located between said coaxial tubular layers.

14. An implantable constriction device according to claim 13, wherein said viscoelastic material is selected from the group consisting of silicone gel, cellulose gel, and collagen gel.

15. An implantable constriction device according to claim 8, wherein said base material forms an outer tubular layer, an inner arcuate layer attached to said outer tubular layer, said outer and inner layers defining a curved space extending longitudinally along said tubing, and said property improving means comprises viscoelastic material filling said space.

16. An implantable constriction device according to claim 15, wherein said viscoelastic material is selected from the group consisting of silicone gel, cellulose gel, and collagen gel.

17. An implantable constriction device according to claim 1, wherein said property improving means comprises a coating on said base material at least along a side of said elongate composite structure that is intended to contact the stomach or esophagus, said coating having better anti-friction properties than said base material.

18. An implantable constriction device according to claim 17, wherein said coating is selected from the group consisting of a Teflon™, Parylene™, and a biocompatible metal coating.

19. An implantable constriction device according to claim 18, wherein the biocompatible metal coating is selected from the group consisting of gold, silver and titanium.

20. An implantable constriction device according to claim 17, wherein said property improving means comprises a core of a viscoelastic material covered with said self-supporting base material.

21. An implantable constriction device according to claim 20, wherein hard silicone constitutes said base material.

22 An implantable constriction device according to claim 20, wherein said viscoelastic material is selected from the group consisting of silicone gel, cellulose gel, and collagen gel.

23. An implantable constriction device according to claim 17, wherein said base material forms an inflatable tubing.

24. An implantable constriction device according to claim 23, wherein said tubing has an inner surface defining the interior of said tubing, and said coating covers said inner surface.

25. An implantable constriction device according to claim 23, wherein said coating is selected from the group consisting of a Teflon™, Parylene™, and a biocompatible metal coating.

26. An implantable constriction device according to claim 25, wherein the biocompatible metal coating is selected from the group consisting of gold, silver and titanium.

27. An implantable constriction device according to claim 23, wherein hard silicone constitutes said base material.

28. An implantable constriction device according to claim 23, wherein said base material forms two coaxial tubular layers and said property improving means comprises a tubular intermediate layer of a viscoelastic material located between said coaxial tubular layers.

29. An implantable constriction device according to claim 28, wherein said viscoelastic material is selected from the group consisting of silicone gel, cellulose gel, and collagen gel.

30. An implantable constriction device according to claim 23, wherein said base material forms an outer tubular layer, an inner arcuate layer attached to said outer tubular layer, said outer and inner layers defining a curved space extending longitudinally along said tubing, and said property improving means comprises a viscoelastic material filling said space.

31. An implantable constriction device according to claim 30, wherein said viscoelastic material is selected from the group consisting of silicone gel, cellulose gel, and collagen gel.

32. An implantable constriction device according to claim 1, wherein said base material forms a first layer and said property improving means comprises a second layer applied on said first layer, said second layer being more fatigue resistant than said first layer.

33. An implantable constriction device according to claim 32, wherein said second layer covers said first layer of said base material along a side of said elongate composite structure that is intended to contact the stomach or esophagus.

34. An implantable constriction device according to claim 32, wherein said second layer comprises a polyurethane layer.

35. An implantable constriction device according to claim 32, wherein said property improving means comprises a coating coated on said first layer and/or said second layer, said coating having better aggressive body fluid resistance properties and/or better anti-friction properties than said base material.

36. An implantable constriction device according to claim 35, wherein said coating is selected from the group consisting of Teflon™, Parylene™, and biocompatible metal coating.

37. An implantable constriction device according to claim 36, wherein the biocompatible metal coating is selected from the group consisting of gold, silver and titanium.

38. An implantable constriction device according to claim 32, wherein hard silicone constitutes said base material.

39. An implantable constriction device according to claim 32, wherein said first layer of said base material forms an inflatable tubing, and said second layer covers said base material within said tubing.

40. An implantable constriction device according to claim 1, wherein said base material forms an inflatable tubing and said property improving means comprises a liquid impermeable coating coated on said base material.

41. An implantable constriction device according to claim 40, wherein said tubing has an external surface of said base material and an internal surface of said base material defining the interior of said tubing, said coating being coated on said external surface and/or internal surface.

42. An implantable constriction device according to claim 40, wherein said coating is selected from the group consisting of Parylene™ and a biocompatible metal coating.

43. An implantable constriction device according to claim 42, wherein the biocompatible metal coating is selected from the group consisting of gold, silver and titanium.

44. An implantable constriction device according to claim 40, wherein hard silicone constitutes said base material.

45. An implantable constriction device according to claim 40, wherein said base material forms two coaxial tubular layers and said property improving means comprises a tubular intermediate layer of a viscoelastic material located between said coaxial tubular layers.

46. An implantable constriction device according to claim 44, wherein said viscoelastic material comprises silicone gel, cellulose gel or collagen gel.

47. An implantable constriction device according to claim 40, wherein said base material forms an outer tubular layer and an inner arcuate layer attached to said outer tubular layer, said outer and inner layers defining a curved space extending longitudinally along said tubing, and said property improving means comprises viscoelastic material filling said space.

48. An implantable constriction device according to claim 47, wherein said viscoelastic material is selected from the group consisting of silicone gel, cellulose gel, and collagen gel.

49. An implantable constriction device according to claim 1, wherein said property improving means comprises gas contained in a multiplicity of cavities formed in said base material to improve the flexibility of said composite structure.

50. An implantable constriction device according to claim 49, wherein said cavities are defined by net structures of said base material.

51. An implantable constriction device according to claim 49, wherein Teflon™ constitutes said base material.

52. An implantable constriction device according to claim 49, wherein said composite structure forms an inflatable tubing.

53. An implantable constriction device for forming a restricted stoma opening in the stomach or esophagus of a patient, comprising an elongate composite structure adapted to constrict the stomach or esophagus of the patient, wherein the composite structure includes an elongate biocompatible self-supporting base material having surfaces exposed to aggressive body cells, when the constriction device is implanted in the patient, and a cell barrier coating coated on said surfaces to prevent body cells from breaking down the base material.

54. An implantable constriction device according to claim 53, wherein said barrier coating is selected from the group consisting of Parylene™ and a biocompatible metal coating.

55. An implantable constriction device according to claim 54, wherein the biocompatible metal coating is selected from the group consisting of gold, silver and titanium.

56. An implantable constriction device for forming a restricted stoma opening in the stomach or esophagus of a patient, comprising:
elongate means for constricting the stomach or esophagus of the patient, the constricting means including
means for making the constricting means self-supporting, and
means for improving at least one physical property of said constricting means other than self-supporting properties.

57. An implantable constriction device according to claim 56, wherein said property improving means improves the resistance to aggressive body cells or the anti-friction properties of said constricting means.

58. An implantable constriction device according to claim 57, wherein said property improving means comprises a coating on said self-supporting means at least along a side of said elongate constricting means that is intended to contact the stomach or esophagus.

59. An implantable constriction device according to claim 58, wherein said coating is selected from the group consisting of a Teflon™, Parylene™, and a biocompatible metal coating.

60. An implantable constriction device according to claim 59, wherein the biocompatible metal coating is selected from the group consisting of gold, silver and titanium.

61. An implantable constriction device according to claim 56, wherein said property improving means improves the flexibility of said constricting means.

62. An implantable constriction device according to claim 61, wherein said property improving means comprises a core of a viscoelastic material covered with said self-supporting base material.

63. An implantable constriction device according to claim 62, wherein hard silicone constitutes said self-supporting means.

64. An implantable constriction device according to claim 62, wherein said viscoelastic material is selected from the group consisting of silicone gel, cellulose gel, and collagen gel.

65. An implantable constriction device according to claim 61, wherein said property improving means comprises gas contained in a multiplicity of cavities formed in said self-supporting means to improve the flexibility of said constricting means.

66. An implantable constriction device according to claim 65, wherein said cavities are defined by net structures of said self-supporting means.

67. An implantable constriction device according to claim 65, wherein Teflon™ constitutes said self-supporting means.

68. An implantable constriction device according to claim 56, wherein said property improving means improves the fatigue resistance of said constricting means.

69. An implantable constriction device according to claim 68, wherein said self-supporting means forms a first layer and said property improving means comprises a second layer applied on said first layer, said second layer being more fatigue resistant than said first layer.

70. An implantable constriction device according to claim 69, wherein said second layer covers said first layer of said self-supporting means along a side of said elongate constricting means that is intended to contact the esophagus or stomach.

71. An implantable constriction device according to claim 69, wherein said second layer comprises a polyurethane layer.

72. An implantable constriction device according to claim 56, wherein said property improving means improves the liquid impermeability of said constricting means.

73. An implantable constriction device according to claim 72, wherein said self-supporting means forms an inflatable tubing and said property improving means comprises a liquid impermeable coating coated on said self-supporting means.

74. An implantable constriction device according to claim 73, wherein said tubing has an external surface of said self-supporting means and an internal surface of said self-supporting means defining the interior of said tubing, said coating being coated on said external surface and/or internal surface.

75. An implantable constriction device according to claim 73, wherein said coating is selected from the group consisting of Parylene™ and a biocompatible metal coating.

76. An implantable constriction device according to claim 75, wherein the biocompatible metal coating is selected from the group consisting of gold, silver and titanium.

77. An implantable constriction device according to claim 55, wherein hard silicon constitutes said self-supporting means.

78. An implantable constriction device for forming a restricted stoma opening in the stomach or esophagus of a patient, comprising:
an elongate composite structure adapted to constrict the stomach or esophagus of the patient,
a base material of said composite structure making said composite structure self-supporting, and
a coating on said base material at least along a side of said elongate composite structure that is intended to contact the stomach or esophagus, said coating having better aggressive body fluid resistant properties than said base material.

79. An implantable constriction device for forming a restricted stoma opening in the stomach or esophagus of a patient, comprising:
an elongate composite structure adapted to constrict the stomach or esophagus of the patient,
a base material of said composite structure making said composite structure self-supporting, and
a coating on said base material at least along a side of said elongate composite structure that is intended to contact the stomach or esophagus, said coating having better anti-friction properties than said base material.

80. An implantable constriction device for forming a restricted stoma opening in the stomach or esophagus of a patient, comprising:
an elongate composite structure adapted to constrict the stomach or esophagus of the patient,
a base material of said composite structure making said composite structure self-supporting, said base material forming a first layer, and
a second layer applied on said first layer, said second layer being more fatigue resistant than said first layer.

81. An implantable constriction device for forming a restricted stoma opening in the stomach or esophagus of a patient, comprising:
an elongate composite structure adapted to constrict the stomach or esophagus of the patient,
a liquid semi-permeable base material of said composite structure forming an inflatable tubing and making said composite structure self-supporting, and
a liquid impermeable coating coated on said base material.

82. An implantable constriction device for forming a restricted stoma opening in the stomach or esophagus of a patient, comprising:
an elongate composite structure adapted to constrict the stomach or esophagus of the patient, and
a base material of said composite structure making said composite structure self-supporting, said base material forming a multiplicity of gas-containing cavities to improve the flexibility of said composite structure.
